(19) United States
(12) Patent Application Publication
(10) Pub. No.: US 2008/0131954 A1
(43) Pub. Date: Jun. 5, 2008

(54) Method of Separating Target DNA from Mixed DNA

(75) Inventor: Michele R. Stone, Rockville, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., Rockville, MD (US)

(21) Appl. No.: 11/946,305

(22) Filed: Nov. 28, 2007

Related U.S. Application Data
(60) Provisional application No. 60/867,855, filed on Nov. 30, 2006.

(51) Int. Cl. C12N 1/08; C07H 21/04

(52) U.S. Cl. 435/270; 536/25.4

(57) ABSTRACT

The present invention relates to methods of separating target DNA from mixed DNA in a sample. In some embodiments, the target DNA may be viral DNA, prokaryotic DNA, fungal DNA or combinations thereof. In some embodiments the mixed DNA includes target DNA and non-target DNA.

This application claims the benefit of Provisional Patent Application No. 60/867,855, filed on Nov. 30, 2006, which is incorporated herein by reference.

### BACKGROUND

1. Field of the Invention

The present invention relates to methods of separating target DNA from mixed DNA in a sample. In some embodiments, the target DNA may be viral DNA, prokaryotic DNA, fungal DNA or combinations thereof. In some embodiments the mixed DNA includes target DNA and non-target DNA.

2. Description of Related Art

The detection of nucleic acids is central to medicine. The ability to detect infectious organisms (e.g., viruses, bacteria, fungi) is ubiquitous technology for disease diagnosis and prognosis. Determination of the integrity of a nucleic acid of interest can be relevant to the pathology of an infection. One of the most powerful and basic technologies to detect small quantities of nucleic acids is to replicate some or all of a nucleic acid sequence many times, and then analyze the amplification products. PCR is perhaps the most well-known of a number of different amplification techniques. The nucleic acids are generally isolated from a sample prior to detection, although in situ detection can also be performed.

The basic steps of nucleic acid, such as DNA, isolation are disruption of the cellular structure to create a lysate, separation of the soluble nucleic acid from cell debris and other insoluble material, and purification of the DNA of interest from soluble proteins and other nucleic acids.

Historically, organic extraction (e.g., phenol:chloroform) followed by ethanol precipitation was done to isolate DNA. Disruption of most cells is done by chaotropic salts, detergents or alkaline denaturation, and the resulting lysate is cleared by centrifugation, filtration or magnetic clearing. The DNA can then be purified from the soluble portion of the lysate. When silica matrices are used, the DNA is eluted in an aqueous buffer such as Tris-EDTA (TE) or nuclease-free water.

DNA isolation systems for genomic, plasmid and PCR product purification are historically based on purification by silica. Regardless of the method used to create a cleared lysate, the DNA of interest can be isolated by virtue of its ability to bind silica in the presence of high concentrations of chaotropic salts (Chen and Thomas, Anal Biochem 101:339-341, 1980; Marko et al., Anal Biochem 121:382-387, 1982; Boom et al., J Clin Microbiol 28:495-503, 1990). These salts are then removed with an alcohol-based wash and the DNA eluted in a low ionic strength solution such as TE buffer or water. The binding of DNA to silica seems to be driven by dehydration and hydrogen bond formation, which competes against weak electrostatic repulsion (Melzak et al., J Colloid and Interface Science 181:635-644, 1996). Hence, a high concentration of salt will help drive DNA adsorption onto silica, and a low concentration will release the DNA.

Recently, new methods for DNA purification have been developed which take advantage of the negatively charged backbone of DNA to a positively charged solid substrate (under specific pH conditions), and eluting the DNA using a change in solvent pH (ChargeSwitch® technology, Invitrogen, Corp., Carlsbad, Calif.; see, for example, U.S. Pat. No. 6,914,137 and International Published Application No. 2006/004611). Whatman has an alternate technology (FTA® paper) that utilizes a cellulose based solid substrate impregnated with a lysis material that lyses cells, inactivates proteins, but captures DNA in the cellulose fibers, where it is retained for use in downstream applications (see, for example, U.S. Pat. No. 6,322,983). Regardless of the applications there is no way to use any of the above described technologies to separate viral, bacterial or fungal DNA from mammalian DNA.

Early detection of infectious agents in a mammalian tissue sample, such as whole blood, requires that a few infectious agent DNA molecules be detected in a background of many mammalian tissue DNA molecules. Separation of the infectious agent DNA molecules from the mammalian tissue DNA molecules would improve detection efficiencies by lowering the background of mammalian DNA in the sample. None of the above described methods address the problem of purifying bacterial, viral, or fungal DNA separately from mammalian DNA in a mixed DNA sample. Thus, a need exists for methods that provide for the enrichment and purification of viral, bacterial or fungal DNA in the presence of mammalian DNA.

### SUMMARY OF THE INVENTION

The present invention relates to methods for separating target DNA from non-target DNA in a sample. In some embodiments, the target DNA may be viral DNA, bacterial (or prokaryotic) DNA, fungal DNA or combinations thereof. In some embodiments, the non-target DNA is mammalian DNA.

Thus, in a first aspect, the present invention provides a method of separating target DNA from mixed DNA in a sample comprising: (a) contacting a sample comprising target DNA and non-target DNA with an agent that binds non-target DNA but does not bind target DNA and (b) separating the target DNA from the bound non-target DNA. In some embodiments, the target DNA may be viral DNA, bacterial DNA, fungal DNA and combinations thereof. In some embodiments, the non-target DNA is mammalian DNA. In some embodiments, the sample is contacted with the agent for a length of time sufficient to bind the non-target DNA.

In further embodiments, the method further comprises contacting the sample with bound non-target DNA with a solid substrate that binds the agent prior to separating the target DNA from the non-target DNA. In other embodiments, the sample comprises cells and the method further comprises first lysing the cells before contacting the sample with the agent. In some embodiments, the lysis is performed by chemical lysis. In other embodiments, the lysis is performed by mechanical energy, preferably acoustic energy. In further embodiments, the method further comprises removing cellular debris from the lysed sample prior to contacting with the agent. In additional embodiments, the agent is coupled to a solid substrate. In some embodiments, the separation is performed by removing the solid substrate from the sample. In other embodiments, the separation is performed by eluting the sample from the solid substrate. In some embodiments, the agent that binds non-target DNA is a chromatin-binding molecule or, more specifically, a histone-binding molecule such as an anti-histone antibody, an anti-histone peptide, an anti-histone aptamer or another anti-histone ligand. In other embodiments, the agent is coupled to the solid substrate by an anti-agent antibody. In some embodiments, the solid substrate is a magnetic bead, a particle, a polymeric bead, a chromotagraphic resin, filter paper, a membrane or a hydrogel.

In a second aspect, the present invention provides a method of separating target DNA from mixed DNA in a cellular sample comprising: (a) lysing the cells of a cellular sample comprising target DNA and non-target DNA, (b) removing cellular debris from the lysed sample, (c) contacting the lysed sample with an agent that binds non-target DNA but does not bind target DNA, and (d) separating the target DNA from the bound non-target DNA. In some embodiments, the target DNA may be viral DNA, bacterial DNA, fungal DNA and combinations thereof. In other embodiments, the non-target DNA is mammalian DNA. In some embodiments, the lysis is performed by chemical lysis. In other embodiments, the lysis is performed by mechanical energy, preferably acoustic energy. In other embodiments, the sample is contacted with the agent for a length of time sufficient to bind the non-target DNA.

In further embodiments, the method further comprises contacting the sample with bound non-target DNA with a solid substrate that binds the agent prior to separating the target DNA from the non-target DNA. In additional embodiments, the agent is coupled to a solid substrate. In some embodiments, the separation is performed by removing the solid substrate from the sample. In some embodiments, the agent that binds non-target DNA is a chromatin-binding molecule or, more specifically, a histone-binding molecule such as an anti-histone antibody, an anti-histone peptide, an anti-histone aptamer or another anti-histone ligand. In other embodiments, the agent is coupled to the solid substrate by an anti-agent antibody. In some embodiments, the solid substrate is a magnetic bead, a particle, a polymeric bead, a chromotagraphic resin, filter paper, a membrane or a hydrogel.

The above and other embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements.

FIG. 1 shows an illustration of separating mammalian DNA from bacterial DNA in accordance with an embodiment of the present invention; and

FIG. 2 shows the use of an unfixed cell ChIP assay to remove mammalian DNA from blood.

### DETAILED DESCRIPTION OF THE INVENTION

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger, Principles of Biochemistry 3rd Ed., W. H. Freeman Pub., New York, N.Y. and Berg et al. (2002)Biochemistry, 5th Ed., W. H. Freeman Pub., New York, N.Y., all of which are herein incorporated in their entirety by reference for all purposes.

As described above, there are no methods which address the problem of purifying bacterial, viral, and/or fungal DNA separately from mammalian DNA in a mixed DNA sample. The present invention provides for the enrichment and purification of bacterial, viral and/or fungal DNA in the presence of mammalian DNA. Thus, the present invention relates to methods for separating target DNA from non-target DNA in a mixed DNA sample.

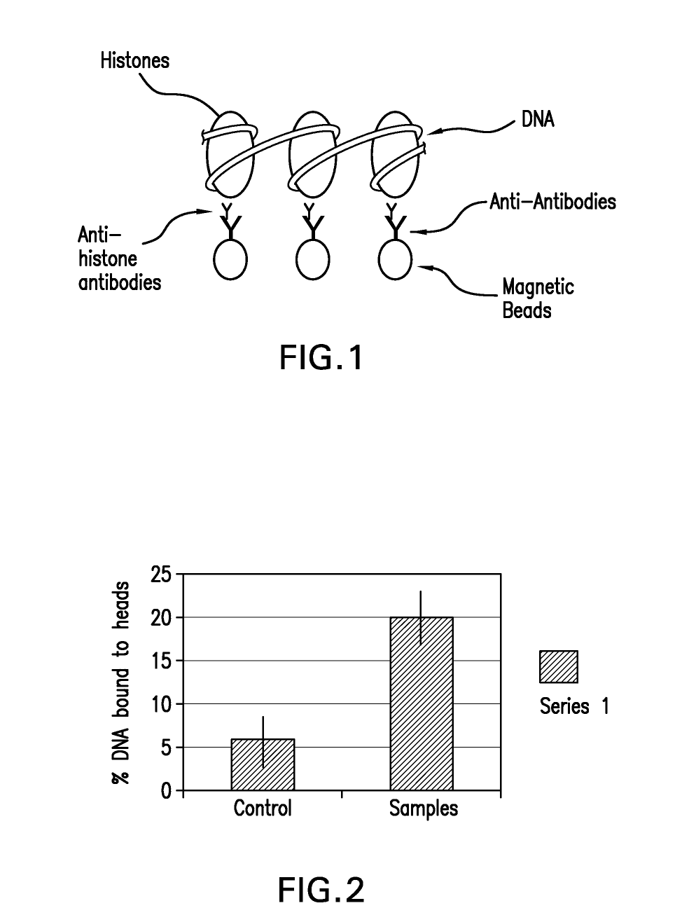

The present invention provides for the separation of non-target DNA, e.g., mammalian DNA, from target DNA, e.g., bacterial, viral and/or fungal DNA, by utilizing the unique characteristic of DNA packaging. DNA is packaged into chromatin. Chromatin is composed of DNA and DNA binding proteins, such as histones. This packaging is illustrated in FIG. 1 which shows that mammalian DNA is wrapped around a histone complex called a nucleosome. The mammalian nucleosome is made up of 4 histone proteins, H2A, H2B, H3 and H4. Mammalian cells use these complexes to compact DNA, forming chromatin. Bacteria and viruses do not have the same histones that mammalian cells have. The present invention takes advantage of this difference to provide for the enrichment for bacterial, viral and/or fungal DNA over mammalian DNA.

Thus, in a first aspect, the present invention provides a method of separating target DNA from mixed DNA in a sample comprising: (a) contacting a sample comprising target DNA and non-target DNA with an agent that binds non-target DNA but does not bind target DNA and (b) separating the target DNA from the bound non-target DNA. In some embodiments, the target DNA is viral DNA, bacterial DNA, fungal DNA or combinations thereof. In some embodiments, the non-target DNA is mammalian DNA. In some embodiments, the sample is contacted with the agent for a length of time sufficient to bind the non-target DNA.

The binding agent is capable of binding to non-target DNA, for example, mammalian DNA, but does not bind to target DNA, for example, viral, bacterial and/or fungal DNA. In one embodiment, the binding agent is a chromatin-binding molecule or, more specifically, a histone-binding molecule such as an anti-histone antibody, an anti-histone peptide, an anti-histone aptamer or another anti-histone ligand. In one embodiment, the binding agent is an antibody (also termed a primary antibody). In one embodiment, the antibody is an antibody that binds to mammalian histones. Examples of anti-histone antibodies include, but are not limited to, rabbit anti-histone 3 (anti-H3) antibody, rabbit anti-H2A antibody, rabbit anti-H2B antibody and rabbit anti-H4 antibody. Anti-histone antibodies are commercially available from Santa Cruz Biotechnology, Inc. (Santa Cruz, Calif., USA) or can be made using conventional techniques well known in the art.

Fragments of antibodies are also useful as binding agents. While various antibody fragments can be obtained by the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain Fv). Single chain antibodies are also useful as a binding agent. Methods for producing single chain antibodies are described in, for example, U.S. Pat. No. 4,946,778. Techniques for the construction of Fab expression libraries are described by Huse et al. (Science 246:1275-1281, 1989); these techniques facilitate rapid identification of monoclonal Fab fragments with the desired specificity. Suitable binding agents also include those that are obtained using methods such as phage display.

In further embodiments, the method further comprises contacting the sample with bound non-target DNA with a solid substrate (also termed a solid phase) that binds the agent prior to separating the target DNA from the bound non-target DNA. In one embodiment, the solid contains an agent that binds the chromatin-binding molecule or, more specifically, a histone-binding molecule such as an anti-histone antibody, an anti-histone peptide, an anti-histone aptamer or another anti-histone ligand. In one embodiment, the solid phase contains a secondary antibody (also termed an anti-antibody) that binds the primary antibody. For example, the primary antibody may be a rabbit anti-H3 antibody and the secondary antibody may be a goat anti-rabbit Ig antibody. In another embodiment, the solid phase is suitable for binding the chromatin-binding molecule or, more specifically, a histone-binding molecule such as an anti-histone antibody, an anti-histone peptide, an anti-histone aptamer or another anti-histone ligand. In some embodiments, the separation is performed by removing the solid substrate from the sample.

In an alternative embodiment, the chromatin-binding molecule or, more specifically, a histone-binding molecule such as an anti-histone antibody, an anti-histone peptide, an anti-histone aptamer or another anti-histone ligand, is bound to a solid substrate and the solid substrate is then contacted with the sample to bind the non-target DNA. In one embodiment, a primary antibody is bound to a solid substrate. As above, the chromatin-binding molecule or, more specifically, a histone-binding molecule such as an anti-histone antibody, an anti-histone peptide, an anti-histone aptamer or another anti-histone ligand may be bound to the solid phase through an agent that binds the chromatin-binding molecule. For example, the primary antibody may be bound to the solid phase through the use of a secondary antibody or through the use of a solid phase which binds the primary antibody. In some embodiments, the separation is performed by removing the solid substrate from the sample.

The “solid substrate” or “solid phase” is not critical and can be selected by one skilled in the art. A “solid phase”, as used herein, refers to any material which is insoluble, or can be made insoluble by a subsequent reaction. Examples of commonly used solid phase materials include, but are not limited to, glass or polymeric tubes which are coated with an antibody on their internal surfaces, coated polymeric inserts, coated polymeric sticks, micro and macro beads formed of polymers and of glass, magnetic beads or particles, porous matrices, coated membranes, tablets, latex particles, microparticles, membranes, plastic tubes, walls of microtiter wells, glass or silicon chips, a chromotagraphic resin, filter paper, a hydrogel and tanned sheep red blood cells are all suitable examples. See, for example, U.S. Pat. Nos. 3,867,517, 3,932,141, 3,951,748, 4,066,512, 4,092,408, 4,255,575, 4,378,344, 4,454,234 and 5,256,561. Among the advantages of solid phase systems is that the reaction product or products can be separated from the reaction solution with relative ease, i.e., by physically removing the solid phase material or by eluting the solution.

Methods for the immobilization of a chromatin-binding molecule or, more specifically, a histone-binding molecule such as an anti-histone antibody, an anti-histone peptide, an anti-histone aptamer or another anti-histone ligand, are well known to those skilled in the art. Suitable methods for immobilizing chromatin-binding molecules, such as antibodies, on solid phases include ionic, hydrophobic, covalent interactions and the like. For example, an antibody may be immobilized by adsorption to a solid phase or by covalent attachment to a solid phase. The manner of coupling an antibody to a solid phase material is known. See, for example, U.S. Pat. Nos. 3,652,761, 3,879,262, 3,896,217, 4,092,408 and 4,378,344. Alternatively, an antibody may be tagged with a small molecule such as biotin and either avidin or an antibody to biotin may be immobilized on a solid phase.

One embodiment of the invention allows for the collection of chromatin using a magnetic bead platform. This methodology is beneficial due to its ease of use and can be adapted to commercial platforms that utilize DNA separation by magnets. The removal of chromatin from the cell lysate allows for the enrichment of bacterial, viral and/or fungal DNA over the background of host DNA. This allows for increased signal to noise ratio in molecular diagnostic assays (example PCR reactions), which is important in cases where it is necessary to detect rare targets, such as bacteria, viruses or fungi.

FIG. 1 is an illustration of this embodiment of the present invention. As shown in FIG. 1, a primary antibody, i.e., an anti-histone antibody (for example, a rabbit anti-histone antibody) is bound to a solid phase, such as a magnetic bead, through the use of a secondary antibody (also termed an anti-antibody) that binds the primary antibody (for example, goat anti-rabbit Ig antibody). For example, a second antibody reactive to the first antibody (such as an anti-antibody raised to the first antibody but in a different animal) bound to an insoluble bead can be used to withdraw the complex from the liquid phase. In this embodiment, the present invention utilizes anti-histone antibodies to immunoprecipitate mammalian DNA from blood sample cell lysates containing non-mammalian DNA.

In other embodiments, the sample comprises cells and the method further comprises first lysing the cells before contacting the sample with the agent. In some embodiments, the lysis is performed by chemical lysis. In other embodiments, the lysis is performed by mechanical energy, preferably acoustic energy. In further embodiments, the method further comprises removing cellular debris from the lysed sample prior to contacting with the agent.

Commercial cell lysis products can be used to lyse cells in the cellular sample. Such commercial cell lysis products include, but are not limited to, Poppers Cell Lysis Reagents (Pierce, Rockville, Ill., USA), Wizard® Genomic DNA Purification Kit (Promega Corp., Madison, Wis., USA), lysis solutions from Qiagen, Inc. (Valencia, Calif., USA), and Cell Lysis Solution (Spectrum Chemical and Laboratory Products, Gardena, Calif., USA).

Alternatively, mechanical energy, preferably acoustic energy, can be used to lyse cells in a cellular sample. Any device that generates a sound wave can be used as a source of acoustic energy for lysing the cells. Such devices include, but are not limited to, ultrasonic transducers, piezoelectric transducers, magnorestrictive transducers and electrostatic transducers. Suitable devices are well known in the art including such commercially available devices as Sonicator 4000 (Misonix, Inc., Farmingdale, N.Y., USA), Microson® Sonicator Microprobe or Micro Cup Horn (Kimble/Kontes, Vineland, N.J., USA) and Covaris™ Adaptive Focused Acoustics (Nexus Biosystems, Poway, Calif., USA). Other suitable devices are described in U.S. Pat. Nos. 6,881,541 and 6,878,540 and in U.S. Patent Application Publication No. 2007/0170812. One advantage of lysing cells using acoustic energy is that not only are the cells lysed, but the chromatin is also sheared to generate fragments of DNA. It is easier for the binding agents, such as antibodies, to interact with the DNA of smaller fragments.

The present invention can be practiced using readily available materials as described above to separate target DNA and non-target DNA in a mixed DNA sample.

In addition, the present invention can be practiced using commercially available reagents and kits. For example, Millipore/Upstate (Lake Placid, N.Y.) has the Chromatin Immunoprecipitation (ChIP) Assay Kit (#17-295), Aviva Systems Biology (San Diego, Calif.) has the ChIP GLAS System (AK-0503), and Active Motif (Carlsbad, Calif.) has the ChIP-It kit (53001). These commercial kits have previously strictly been used to explore in vivo interactions between proteins and DNA, to identify transcription regulations sites, and to identify methylated and un-methylated regions of chromatin. Although these reagent kits are not designed to separate mammalian DNA from mixtures of mammalian and viral, bacterial and/or fungal DNA, they can be adapted for such use.

Experimental evidence shows that this method is useful in eliminating a significant fraction of mammalian DNA (non-target DNA) from a blood sample, and thus provides an enrichment of viral, bacterial and/or fungal DNA (target DNA). An assay was designed using a commercial ChIP assay, from Upstate Biological catalog numbers 17-295 and 17-375 (Millipore/Upstate, Lake Placid, N.Y., USA), along with antibodies purchased from abcam catalog number ab1791 and 46540 (Abcam Inc., Cambridge, Mass., USA) to be able to measure and quantify the removal of mammalian DNA from a sample of sheep whole blood. This assay involved lysing the cells via sonication to liberate internal material as well as sheer the chromatin inside the cell. This generates about 300 bp fragments of DNA that are easy for antibodies to interact with. After lysis, the sample is pelleted in a centrifuge at 13,000 rpm for 10 min at 4° C. to pellet cellular debris. The supernatant is then mixed with a primary antibody to a histone, in this case it as a ChIP grade mouse anti-H3 antibody and incubated overnight for a complete immunoprecipitation. Magnetic beads, coupled to a secondary antibody (goat anti-mouse Ig), were then incubated with the mixture. The magnetic beads were collected using a magnetic field and the DNA co-precipitated with the histone complex on the beads was quantified using a pico-green assay. Preliminary results indicate that this method allows for the immunoprecipitations of mammalian DNA from a blood sample allowing for the specific separation of mammalian DNA from whole blood samples.

FIG. 2 shows that a ChIP assay can be used to remove mammalian DNA from blood. The control bar represents the average of three experiments done in duplicate. The control experiment was run identical to the sample experiment with the exception of a deletion of a primary antibody incubation step. Without the primary antibody there should be only background binding of the histone complex with the goat anti-mouse magnetic beads. The value represents the background value for the method. The sample bar represents the average of three experiments done in duplicate. The bar values are the % DNA bound to the beads and the error bars represent the standard deviation between the three experiments. These results demonstrate that at least 20% of mammalian DNA can be removed from a sample of whole blood. These preliminary results demonstrate that a significant amount of mammalian DNA can be separated away from the target DNA in a mixed DNA sample using the methods of the present invention. Further optimization of the method will increase the percentage removal of mammalian DNA.

In a second aspect, the present invention provides a method of separating target DNA from mixed DNA in a cellular sample comprising: (a) lysing the cells of a cellular sample comprising target DNA and non-target DNA, (b) removing cellular debris from the lysed sample, (c) contacting the lysed sample with an agent that binds non-target DNA but does not bind target DNA, and (d) separating the target DNA from the bound non-target DNA. In some embodiments, the target DNA may be viral DNA, bacterial DNA, fungal DNA and combinations thereof. In other embodiments, the non-target DNA is mammalian DNA. The cells are lysed as described herein or using conventional techniques well known to the skilled artisan. In some embodiments, the lysis is performed by chemical lysis as described herein. In other embodiments, the lysis is performed by mechanical energy, preferably acoustic energy, as described herein. The cellular debris is removed using conventional techniques well known to the skilled artisan. The contacting and separating steps are performed as described herein. In other embodiments, the sample is contacted with the agent for a length of time sufficient to bind the non-target DNA.

In further embodiments, the method further comprises contacting the sample with bound non-target DNA with a solid substrate that binds the agent prior to separating the target DNA from the non-target DNA as described herein. In additional embodiments, the agent is coupled to a solid substrate as described herein. In some embodiments, the separation is performed by removing the solid substrate from the sample as described herein. In some embodiments, the agent that binds non-target DNA is a chromatin-binding molecule or, more specifically, a histone-binding molecule such as an anti-histone antibody, an anti-histone peptide, an anti-histone aptamer or another anti-histone ligand. In one embodiment, the agent that binds non-target DNA is an anti-histone antibody as described herein. In other embodiments, the agent is coupled to the solid substrate by an agent that binds the chromatin-binding molecule. In one embodiment, an anti-agent antibody as described herein is used. In some embodiments, the solid substrate is a magnetic bead, a particle, a polymeric bead, a chromotagraphic resin, filter paper, a membrane or a hydrogel, as described herein.

The current state of the art in molecular diagnostics for infectious disease does not include separation of bacterial, viral and/or fungal DNA from background mammalian DNA in tissue extracts. Instead the mixed sample is utilized for the specific amplification and detection of the target bacterial, viral and/or fungal DNA. In many cases the background mammalian DNA interferes with amplification and detection. The present invention can be used to remove background mammalian DNA prior to the amplification and detection steps of diagnostic procedures for the bacterial, viral and/or fungal DNA.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of separating target DNA from non-target DNA comprising:
contacting a sample comprising target DNA and non-target DNA with an agent that binds non-target DNA but does not bind target DNA, wherein the target DNA is selected from the group consisting of viral DNA, bacterial DNA, fungal DNA and combinations thereof; and
separating the target DNA from the bound non-target DNA.

2. The method of claim 1, wherein the agent is coupled to a solid substrate.

3. The method of claim 2, wherein the separation is performed by removing the solid substrate from the sample.

4. The method of claim 1, which further comprises contacting the sample with bound non-target DNA with a solid substrate that binds the agent prior to separating the target DNA from the non-target DNA.

5. The method of claim 4, wherein the separation is performed by removing the solid substrate from the sample.

6. The method of claim 1, wherein the agent that binds the non-target DNA is a chromatin-binding molecule.

7. The method of claim 6, wherein the chromatin-binding molecule is selected from the group consisting of a histone-binding molecule, an anti-histone antibody, an anti-histone peptide, an anti-histone aptamer or anti-histone ligand.

8. The method of claim 7, wherein the chromatin-binding molecule is an anti-histone antibody.

9. The method of claim 2, wherein the agent is coupled to the solid substrate by an anti-agent antibody.

10. The method of claim 2, wherein the solid substrate is a magnetic bead, a particle, a polymeric bead, a chromotagraphic resin, filter paper, a membrane or a hydrogel.

11. The method of claim 10, wherein the solid substrate is a magnetic bead.

12. The method of claim 4, wherein the agent that binds the non-target DNA is a chromatin-binding molecule.

13. The method of claim 12, wherein the chromatin-binding molecule is selected from the group consisting of a histone-binding molecule, an anti-histone antibody, an anti-histone peptide, an anti-histone aptamer or anti-histone ligand.

14. The method of claim 13, wherein the chromatin-binding molecule is an anti-histone antibody.

15. The method of claim 4, wherein the solid substrate is a magnetic bead, a particle, a polymeric bead, a chromotagraphic resin, filter paper, a membrane or a hydrogel.

16. The method of claim 15, wherein the solid substrate is a magnetic bead.

17. The method of claim 1, wherein the sample comprises cells and the method further comprises first lysing the cells before contacting the sample with the agent.

18. The method of claim 17, wherein the lysis is performed by chemical lysis.

19. The method of claim 17, wherein the lysis is performed by mechanical energy.

20. The method of claim 19, wherein the lysis is performed by acoustic energy.

21. The method of claim 17, which further comprises removing cellular debris from the lysed sample prior to contacting with the agent.

22. The method of claim 1, wherein the sample is contacted with the agent for a length of time sufficient to bind the non-target DNA

23. The method of claim 1, wherein the non-target DNA is mammalian DNA.

24. A method of separating target DNA from non-target DNA in a cellular sample comprising:
lysing the cells of a cellular sample comprising target DNA and non-target DNA, wherein the target DNA is selected from the group consisting of viral DNA, bacterial DNA, fungal DNA and combinations thereof;
removing cellular debris from the lysed sample;
contacting the lysed sample with an agent that binds non-target DNA but does not bind target DNA; and
separating the target DNA from the bound non-target DNA.

25. The method of claim 24, wherein the agent is coupled to a solid substrate.

26. The method of claim 25, wherein the separation is performed by removing the solid substrate from the sample.

27. The method of claim 24, wherein the agent that binds the non-target DNA is a chromatin-binding molecule.

28. The method of claim 27, wherein the chromatin-binding molecule is selected from the group consisting of a histone-binding molecule, an anti-histone antibody, an anti-histone peptide, an anti-histone aptamer or anti-histone ligand.

29. The method of claim 28, wherein the chromatin-binding molecule is an anti-histone antibody.

30. The method of claim 24, which further comprises contacting the sample with bound non-target DNA with a solid substrate that binds the agent prior to separating the target DNA from the non-target DNA.

31. The method of claim 30, wherein the separation is performed by removing the solid substrate from the sample.

32. The method of claim 30, wherein the agent that binds the non-target DNA is a chromatin-binding molecule.

33. The method of claim 32, wherein the chromatin-binding molecule is selected from the group consisting of a histone-binding molecule, an anti-histone antibody, an anti-histone peptide, an anti-histone aptamer or anti-histone ligand.

34. The method of claim 33, wherein the chromatin-binding molecule is an anti-histone antibody.

35. The method of claim 25, wherein the agent is coupled to the solid substrate by an anti-agent antibody.

36. The method of claim 25, wherein the solid substrate is a magnetic bead, a particle, a polymeric bead, a chromotagraphic resin, filter paper, a membrane or a hydrogel.

37. The method of claim 36, wherein the solid substrate is a magnetic bead.

38. The method of claim 36, wherein the solid substrate comprises an anti-agent antibody.

39. The method of claim 30, wherein the solid substrate is a magnetic bead, a particle, a polymeric bead, a chromotagraphic resin, filter paper, a membrane or a hydrogel.

40. The method of claim 39, wherein the solid substrate is a magnetic bead.

41. The method of claim 24, wherein the sample is contacted with the agent for a length of time sufficient to bind the non-target DNA

42. The method of claim 24, wherein the non-target DNA is mammalian DNA.
